(19) United States
(12) Patent Application Publication
(10) Pub. No.: US 2004/0043061 A1
(43) Pub. Date: Mar. 4, 2004

(54) Dissolvable films comprising suspended, non-soluble pharmaceutically active ingredients, apparatus and methods for their manufacture and use

(76) Inventors: Daniel S. Leon, Oyster Bay, NY (US); Paul Gabel, Oyster Bay, NY (US)

Correspondence Address: Galgano & Burke, Suite 35, 300 Rabro Drive, Hauppauge, NY, 11788, US

(21) Appl. No.: 10/417,432

(22) Filed: Apr. 16, 2003

Related U.S. Application Data
(63) Continuation of application No. 09/662,301, filed on Sep. 15, 2000, now abandoned.

(51) Int. Cl. A61K 9/70

(52) U.S. Cl. 424/443

(57) ABSTRACT

Pharmaceutical delivery systems in the form of pliable films comprising a binding agent, a lubricant, a solvent for the binding agent and lubricant, and at least one pharmaceutically active agent which is not soluble in the solvent.

Another aspect of the present invention comprises methods for manufacturing dissolvable films comprising suspended, non-soluble pharmaceutically active ingredients and apparatus useful for the manufacture of these dissolvable films.

Other aspects comprise apparatus for administering the dissolvable films which comprise positioning at least one dissolvable film within a body cavity and methods of administering pharmaceutically active ingredients wherein a dissolvable film is at least partially dissolved and then administered to a patient.

The present invention is directed to delivery systems for certain health care related, effective agents and, more particularly to dissolvable films comprising suspended, non-soluble pharmaceutical ingredients, apparatus and methods for their manufacture and use.

### BACKGROUND

The delivery of pharmaceutical components which are non-soluble, i.e. pharmaceutical suspensions, have posed disadvantages in the past. For example, pharmaceutical suspensions such as cephalosporins, dicholorophene and tetrasodium pyrophosphate have very short shelf lives. Dosages delivered to medical facilities such as doctor's offices that are not administered during their short shelf lives, e.g. several months, are typically returned to the pharmaceutical manufacturer for a credit and disposal. Such suspensions are typically delivered either orally or to patients as injections which inherently cause patient discomfort. It is very expensive to manufacture and ship pharmaceutically active suspensions which are intended to be injected into the body, due in part to their relatively short shelf stability and a common requirement that the dosages remain refrigerated prior to use.

Other pharmaceutical suspensions such as Megestrol, Clarithromycin, Erythromycin, neomycin-polymyxin B-hydrocortisone, Cefuroxime-Axetil pwd, Sulfamethoxazole-trimethoprim, Atovaquone, Azithromycin, Cefpodoxime-Proxetil, Amoxicillin-Clavulanate Acyclovir, Mesalamine enema, Acetominophen-pseudoephedrine-dextromethorphan-chlorpheniramine, Phenylephrine-Chlorpheniramine-Pyrilamine, Leuprolide Acedtate, Acetominophen-codeine phosphate, Ibuprofen, and Colfosceril palmitate-cetyl alcohol-tyloxapol have traditionally been delivered to patients through the gastro-intestinal tract in the forms of digestible liquids or are provided in spray form. Those skilled in the art will appreciate the limitations that delivery through the gastro-intestinal tract places upon pharmaceuticals. For example, the high acidity of the gastro-intestinal tract can adversely impact the efficacy of the pharmaceutically active agent.

Other known disadvantages have included the need to mix multiple dilutions of active agents with a diluent, some difficulty in swallowing oral dosages, and relatively slow absorptions.

The use of dissolvable films to deliver the contraceptive nonoxynol-9, has been known. Typically such films comprise a binder such as polyvinyl alcohol, a lubricant such as glycerin, a solvent for the polyvinyl alcohol and glycerin such as water, the active ingredient nonoxynol-9 and at least one preservative. Water is utilized as a solvent since the nonoxynol-9 is water soluble and can be readily dissolved into a mixture or slurry prior to formation of the film. The use of dissolvable films to deliver difficult, non-soluble pharmaceutically active compounds has not previously been suggested, to the knowledge of the present inventors.

It would therefore be desirable to provide a delivery system for pharmaceutically active components which are not soluble in generally acceptable pharmaceutical solvents such as water ethanol, glycerin or propylene glycol.

It would also be desirable to provide delivery systems for pharmaceutically active components which are not soluble in such solvents, where the delivery system can have significantly greater shelf stability.

It would also be desirable to provide such pharmaceutically active delivery systems which can be readily administered without significant patient discomfort.

### SUMMARY OF THE INVENTION

Various aspects of the present invention comprise pharmaceutical delivery systems in the form of pliable films comprising a binding agent, a lubricant, a solvent for the binding agent and lubricant, and at least one pharmaceutically active agent which is not soluble in the aforementioned solvent. The films of various embodiments of the present invention advantageously have a lower moisture content which will tend to lengthen the shelf stability of these delivery systems compared to traditional water-based or ethanol-based liquid suspensions. Since the active agents are not maintained in systems in which they are soluble, there is a much less tendency for degradation of the active agent as compared to traditional water-based suspensions.

Another aspect of the present invention comprises methods for manufacturing dissolvable films comprising suspended, non-soluble pharmaceutically active ingredients and apparatus useful for the manufacture of these dissolvable films. As used herein, the term “non-soluble” when used in connection with pharmaceutically active ingredients indicates that the active ingredient is not soluble in the other components used in a given film.

Other embodiments of the present invention comprise methods of administering pharmaceutically active ingredients wherein a dissolvable film comprising at least one non-soluble pharmaceutically active ingredient is administered to a patient orally, topically, intravenously, intramuscularly, vaginally, anally, transdermally, internally, e.g., at the site of an operation, or otherwise placed within or on a patient.

A still further aspect of the present invention comprises an apparatus for administering the dissolvable films which comprise positioning at least one dissolvable film within a body cavity.

According to still further methods of administering pharmaceutically active ingredients in accordance with the current invention, a dissolvable film comprising at least one non-soluble pharmaceutically active ingredient is at least partially dissolved and then administered to a patient orally, as a vapor, topically, intravenously, intramuscularly, vaginally, anally, transdermally, or internally, e.g., at the site of an operation, or otherwise placed within or on a patient.

These and other aspects of the present invention are described in further detail below.

### BRIEF DESCRIPTION OF THE DRAWINGS

FIG. 1 is a schematic illustration of one method of manufacturing dissolvable films of the present invention.

FIG. 2 is a cross-sectional illustration of an extrusion head of the prior art.

FIG. 3 is a cross-sectional illustration of an extrusion head of one embodiment of the present invention.

FIG. 4 is an illustration showing a mixer disposed within the first chamber of an extrusion head of one embodiment of the present invention.

FIG. 5 is a closeup view of the second chamber of the extrusion head cavity illustrated in FIG. 3.

FIG. 6 illustrates a storage/delivery device of one embodiment of the present invention.

### DETAILED DESCRIPTION

One embodiment of the present invention comprises a pliable film comprising at least one binding agent, at least one lubricant, a solvent for the binding agent and the lubricant, and an effective amount of at least one pharmaceutically active ingredient. According to preferred embodiments of the present invention, the films are hydrophilic.

One embodiment of the present invention comprises a pliable film comprising at least one binding agent, at least one lubricant, a solvent for the binding agent and the lubricant, and an effective amount of at least one non-soluble pharmaceutically active component. The binding agent which provides a plasticizer effect may comprise polyvinyl alcohol, methylcellulose compounds such as hydroxypropyl, methylcellulose(HPMC) or other gelatin or starch-based binders either alone or in combination. The final film preferably comprises about 40% to 80% of the binding agent and most preferably about 50% to 70% by weight. Unless otherwise noted herein, all percentages noted herein are by weight.

The lubricant can comprise one or more of glycerin, propylene glycol, polyethylene glycols, fish oil, vegetable oil, and combinations thereof. The lubricant is preferably present in the amount of about 2% to 15% and most preferably about 4 to 8% in the final film.

In order to properly blend the binding agent and lubricant, a solvent such as water or an organic solvent such as ethanol is preferably employed. The initial formulation, preferably comprises about 2% to 15% of solvent and most preferably about 3% to 7%. If desired, more than one solvent can be used.

The pharmaceutically active component of the present invention can comprise a wide range of pharmaceuticals such as anti-biotics, pain relievers, anti-inflammatory agents, anti-viral agents, vaccines, nutritional supplements, hypothyroidism medication, anticoagulants, antihistamines, antitussives, hormones, coagulants, antipiretics, and combinations thereof. Specific pharmaceutical actives which can be utilized with the present invention include Megestrol, Clarithromycin, Erythromycin, neomycin-polymyxin B-hydrocortisone, Cefuroxime-Axetil pwd, Sulfamethoxazole-trimethoprim, Atovaquone, Azithromycin, Cefpodoxime-Proxetil, Amoxicillin-Clavulanate Acyclovir, Mesalamine enema, Acetominophen-pseudoephedrine-dextromethorphan-chlorpheniramine, Phenylephrine-Chlorpheniramine-Pyrilamine, Leuprolide Acedtate, Acetominophen-codeine phosphate, Ibuprofen, Colfosceril palmitate-cetyl alcohol-tyloxapol. The amount of the active ingredient employed in the initial mixture will depend upon the desired dosage. According to preferred embodiments of the present invention, the pharmaceutically active component is preferably homogeneously distributed throughout the film, but it is also within the scope to the present invention to provide a non-homogeneous distribution.

Various embodiments of the present invention can also comprise flavorants and/or sweeteners such as aspartame, sorbitol, and flavorants such as mints, fruit flavorants, etc.

It is also in the scope of the present invention to include preservatives such as methyl paraben, propylparaben, ethylenediamine-tetraacetic acid (EDTA), diazolidnyl urea, stearic acid, benzoic acid, sodium benzoate and combinations thereof in order to enhance shelf stability and prevent microbial contamination.

The drug delivery systems of the present invention can advantageously be manufactured on a bulk scale. In order to achieve the desired concentration of pharmaceutically active component in the dosage form, preferred embodiments are manufactured on a weight by weight concentration of the total product.

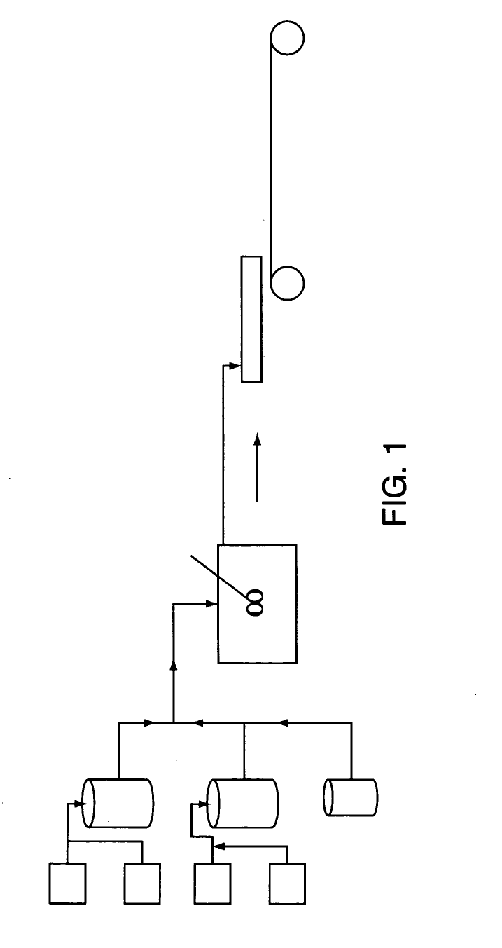

FIG. 1 is a schematic view of a manufacturing process which can be utilized with the present invention. In accordance with this schematic illustration, all ingredients are individually weighed or otherwise measured and combined in a series of small tanks. These pre-mixes may be formed as an emulsion or as solutions depending on the solubility of the ingredients. Typically, these pre-mixes may require heating and constant agitation depending upon the solubility, temperature and mixing characteristics of the components. The pre-mixes may also be combined into larger pre-mixes prior to combining in a final mixing tank. The ingredients are typically mixed and heated separately until the homogenous solution is achieved in each assembly prior to transfer to the final tank. The pharmaceutical active ingredient(s) may be added to either one of the pre-mixes or simply to the final mixing tank. The final mixture comprising the suspended active ingredient is then preferably transferred to an extruder which continues to mix the fluid mixture prior to extrusion.

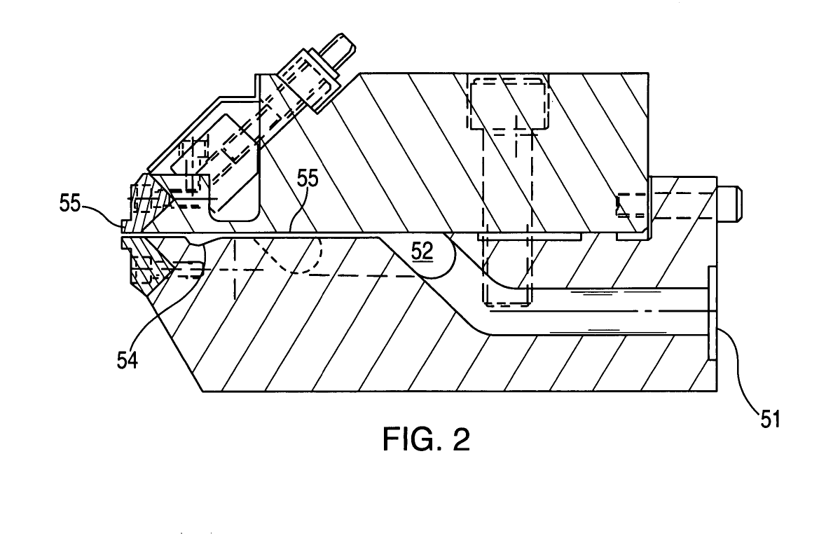

FIG. 2 illustrates an extrusion head 50 of the prior art wherein fluid is pumped into inlet 51, expands in a chamber 52, extending substantially the entire width of the extrusion head and is forced through channel 53 into a single chamber 54 and out through extrusion dye 55. Those skilled in the art will appreciate that the size of the opening 55 of extrusion head 50 is adjustable in order to provide extrusions of different thicknesses.

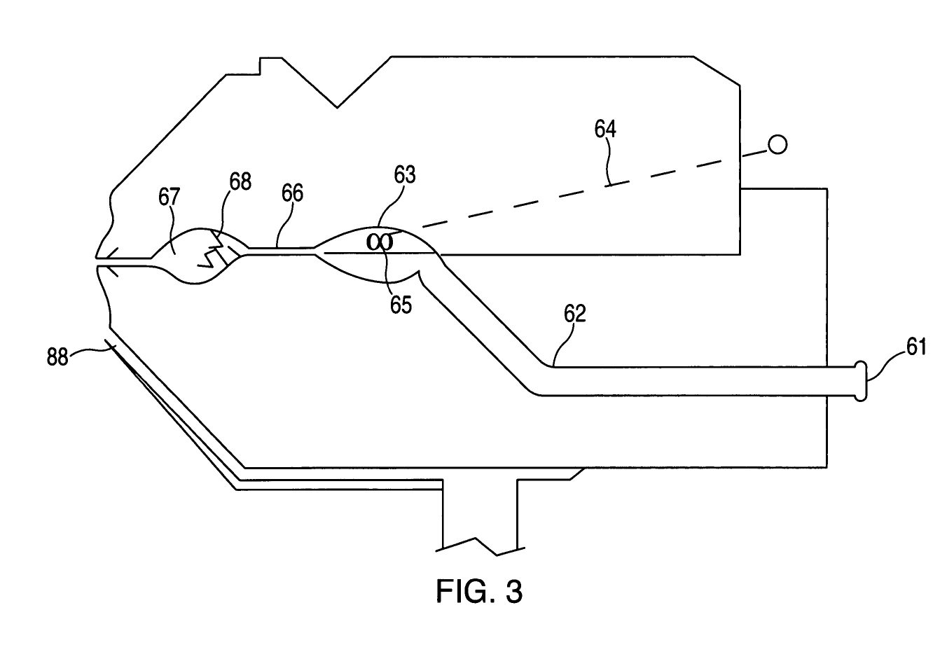

FIG. 3 illustrates an extrusion head of one embodiment of the present invention. As illustrated, the fluid mixture is pumped into inlet 61 through tube 62 into a first chamber 63. A pneumatic shaft 64 having an impeller is disposed within the first chamber 63 in order to preferably continually mix the fluid mixture to prevent the non-soluble pharmaceutically active ingredient from settling or otherwise separating from the mixture. While subject to this mixing, the fluid mixture proceeds through conduit 66 into a second chamber comprising a plurality of baffles 68. The baffles serve to further enhance the intermixing of the ingredients and also to help separate air from the mixture. A vacuum (not shown) may be provided in the first or second chamber, or both, in order to draw off air intermixed with the fluid during pumping and/or the mixing. The fluid mixture is then pumped through the extrusion head outlet 80, preferably onto a moving belt (not shown). As illustrated, a vacuum channel 88 provides a downwardly directed vacuum from a position below the extrusion head outlet 80 in order to draw the sheet down toward the moving belt. The extrusion head illustrated in FIG. 3 comprises all of the adjustments of previously known extrusion heads including adjustments to the width of the head outlet, as well as suitable controls for the pneumatic shafts and vacuums, though these controls are not illustrated in FIG. 3.

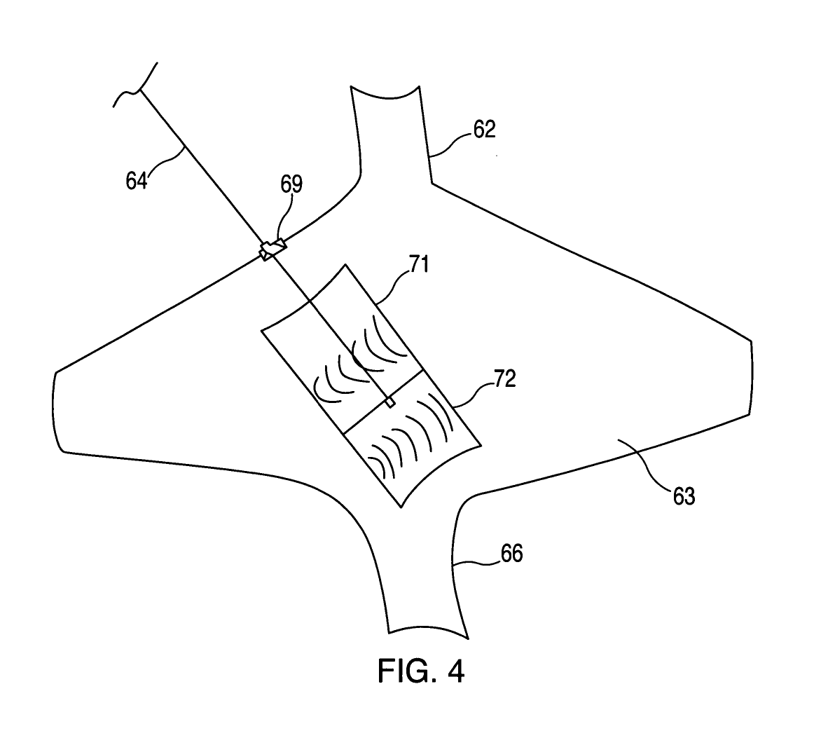

FIG. 4 is a top view of the first chamber showing the inlet tube 62, the first chamber 63 and the outlet tube 66. In addition, a pneumatic shaft 64 is partially illustrated which enters chamber 63 through a sealed bearing 69. A pneumatically operated shaft is preferred in order to minimize the risk of electrical sparks which could be dangerous with certain ingredients used to form the films of the present invention. Attached to pneumatic shaft 64 in this illustrated embodiment is a dual stage processing impeller 63 comprising a first section 71 having impellers angled in a first direction and at least one other section 72 with impellers angled in another direction. In the illustrated embodiment, the dual stage processing impeller 65 is designed for counter-clockwise rotation in order to direct the fluid mixture containing the non-soluble active ingredient toward the outlet.

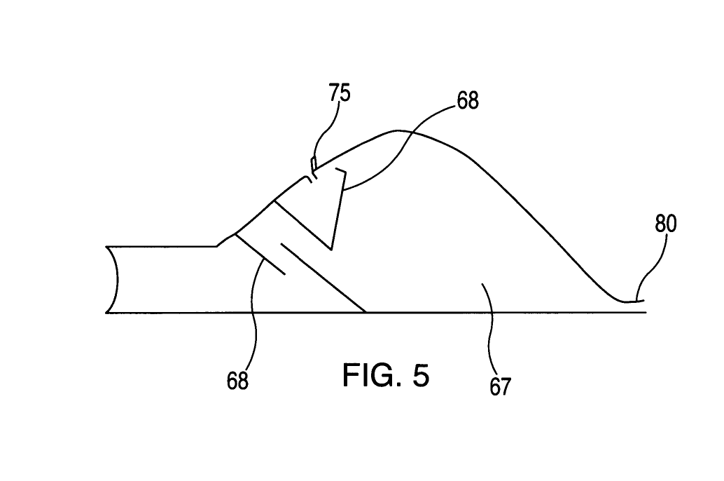

FIG. 5 is a close-up view of the second chamber 67 shown in FIG. 3 wherein a plurality of baffle plates 68 cause the fluid to pass through a tortuous path in order to enhance and continue mixing the non-soluble pharmaceutically active ingredient(s) with the film base fluid mixture. FIG. 5 also shows one preferred positioning of the vacuum port 75 used for drawing off excess air.

In accordance with one preferred embodiment of the present invention, while the pharmaceutically active ingredient is non-soluble in the solvent used to form the film, the film itself is dissolvable in a body cavity. Therefore, methods of the present invention include administering a pharmaceutically active ingredient wherein a dissolvable film comprising at least one non-soluble pharmaceutically active ingredient is administered to a patient orally, topically, intravenously, intramuscularly, vaginally, anally, transdermally, internally, e.g., at the site of an operation, or otherwise placed within or on a patient in a manner which results in the film dissolving.

According to additional aspects of the present invention, methods are provided for administering pharmaceutical actives to a patient comprising the steps of: providing a film comprising at least one binding agent, at least one lubricant, at least one solvent for the binding agent and the lubricant, and an effective amount of at least one pharmaceutically active component which is not soluble in the solvent; dissolving said film in at least one solvent in which the pharmaceutically active agent is soluble; and administering the resultant solution to a patient. According to these methods of the present invention, the resulting solution can be administered to a patient orally, as a vapor, topically, intravenously, intramuscularly, or otherwise placed within or on a patient, such as within a body cavity.

For example, one or more of the suspension films of the present invention can be utilized to replace a traditional dosage of an injectable solution. A suspension film of the present invention can be sent to either a physician or a pharmacist. By simply adding sterile water and/or another acceptable solvent to the film, the film can be returned to a liquid for injection or oral application.

According to a still further embodiment, a delivery device of the present invention, comprises utilizing a pouch having a plurality of compartments. One compartment comprises a suspension film of the present invention, while another compartment comprises a solvent for the suspension film, for example, sterile water, which is separated from the suspension film by a membrane which is not permeable to the water or whatever other solvent is utilized. The membrane would be readily rupturable, for example, by the application of pressure or utilizing an internal rupturing device. Once the membrane has been ruptured, the solvent, e.g., sterile water, mixes with the suspension film thereby providing a liquid suspension. The pouch could then be penetrated by a syringe to extract the medication for use.

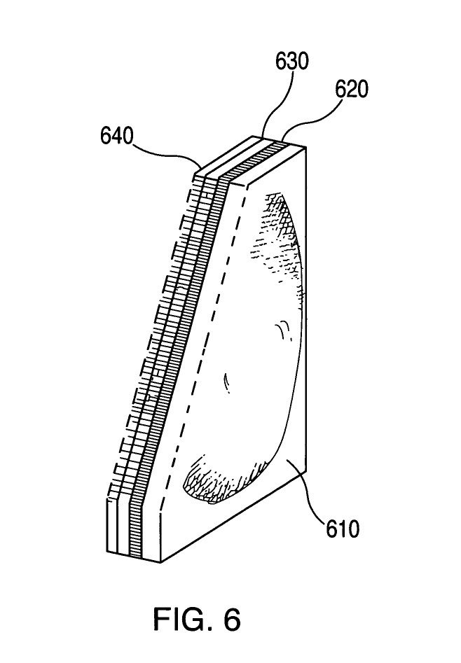

For example, as shown in one embodiment which is illustrated in cross-section in FIG. 6, a storage/delivery device is formed with four layers, a top foil layer 610, a water impermeable membrane 620, a suspension film of the present invention and a lower foil layer 640. As illustrated, water is disposed between the top foil layer and the impermeable membrane. During storage, the water is therefore maintained separate from the suspension film. When it is desired to use the active ingredient in the film, pressure is applied to the bulge in the top of the foil pack thereby applying pressure to the membrane. The membrane is designed to perforate but not fragment. Upon perforation of the membrane, the water contacts the suspension film and forms a liquid suspension. A physician or other medical personnel can then simply insert a needle into the pouch and extract the suspension for injection. Alternatively, the foil portion of the pouch can be opened for application in some other manner. FIG. 6 is purely for illustration purposes. The thickness of each layer is not drawn to scale.

The use of the suspension films of the present invention offer the advantage of, in some cases, longer shelf stability since the active ingredient is not in contact with a liquid solvent during transportation and storage. The use of the disclosed pouches also avoids the use of breakable glass vials and the inherent risk of using such vials in the trauma field. Additionally, embodiments of the present invention will not require refrigeration and, therefore, result in reduced costs to health professionals.

1. A film comprising a suspension of a pharmaceutically active component comprising:
at least one binding agent;
at least one lubricant;
at least one solvent for said binding agent and said lubricant; and
an effective amount of at least one pharmaceutically active component which is not soluble in said solvent.

2. A film comprising a suspension of a pharmaceutically active component according to claim 1 wherein said pharmaceutically active component is homogeneously distributed throughout said film.

3. A film comprising a suspension of a pharmaceutically active component according to claim 1 wherein said lubricant is selected from the group consisting of glycerin, propylene glycol, polyethylene glycols, fish oil, vegetable oil and combinations thereof.

4. A film comprising a suspension of a pharmaceutically active component according to claim 1 wherein said binding agent comprises polyvinyl alcohol.

5. A film comprising a suspension of a pharmaceutically active component according to claim 1 wherein said binding agent comprises hydroxy propyl methyl cellulose.

6. A film comprising a suspension of a pharmaceutically active component according to claim 1 wherein said binding agent is selected from the group consisting of cellulose compounds, gelatins, starches, polyvinyl alcohol, and combinations thereof.

7. A film comprising a suspension of a pharmaceutically active component according to claim 1 wherein said solvent comprises water.

8. A film comprising a suspension of a pharmaceutically active component according to claim 1 wherein said solvent comprises an organic solvent.

9. A film comprising a suspension of a pharmaceutically active component according to claim 8 wherein said solvent comprises ethanol.

10. A film comprising a suspension of a pharmaceutically active component according to claim 1 wherein said film further comprises at least one preservative.

11. A film comprising a suspension of a pharmaceutically active component according to claim 10 wherein said preservative is selected from the group consisting of methylparaben, propylparaben, ethylenediamine-tetra acetic acid (EDTA), diazolidnyl urea, stearic acid, benzoic acid, sodium benzoate and combinations thereof.

12. A film comprising a suspension of a pharmaceutically active component according to claim 1 wherein said film further comprises a flavorant.

13. A film comprising a suspension of a pharmaceutically active component according to claim 12 wherein said flavorant is selected from the group consisting of genuine and artificial fruits, beef, chicken, liver, bacon, cheese, apple, mint and fish flavorants, and combinations thereof.

14. A film comprising a suspension of a pharmaceutically active component according to claim 1 wherein said film further comprises a filler.

15. A film comprising a suspension of a pharmaceutically active component according to claim 14 wherein said filler is selected from the group consisting of cornmeal, cornstarch, potato starch, bone meal, corn syrup, wheat germ, brewers yeast, xanthan gum, carrageenan and combinations thereof.

16. A film comprising a suspension of a pharmaceutically active component according to claim 1 wherein said pharmaceutically active component is selected from the group consisting of antibiotics, pain relievers, anti-inflammatory agents, antiviral agents, vaccines, dietary supplements [would this cover Colfosceril palmitate-cetyl alcohol-tyloxapol ?], hypothyroidism medication, anorexia medications, colitis-proctitis medications, cough suppressants, cold medications, decongestants, anti-histamines, endometriosis medications, and combinations thereof.

17. A film comprising a suspension of a pharmaceutically active component according to claim 1 further comprising at least one pharmaceutically active component which is soluble in said solvent.

18. A film comprising a suspension of a pharmaceutically active component according to claim 1 comprising a plurality of pharmaceutically active components.

19. A film comprising a suspension of a pharmaceutically active component according to claim 1 wherein said film comprises a moisture content of about 1-9% by weight.

20. A film comprising a suspension of a pharmaceutically active component according to claim 1 wherein said film comprises a moisture content of about 4-6% by weight.

21. A method of manufacturing a film comprising a pharmaceutically active component comprising the steps of:
providing at least one binding agent;
providing at least one lubricant;
providing at least one solvent for said binding agent and said lubricant;
combining said binding agent, said lubricant and said solvent to form a fluid mixture;
suspending at least one pharmaceutically active component, which is not soluble in said solvent, in said fluid mixture; and converting said fluid mixture into a film.

22. A method of manufacturing a film comprising a pharmaceutically active component according to claim 21 wherein said step of converting said fluid mixture into a film comprises forming said fluid mixture into a sheet.

23. A method of manufacturing a film comprising a pharmaceutically active component according to claim 22 wherein said step of converting said fluid mixture into a film further comprises the step of removing solvent from said sheet to form a pliable film.

24. A method of manufacturing a film comprising a pharmaceutically active component according to claim 23 wherein said step of removing solvent comprises evaporating solvent from said sheet.

25. A method of manufacturing a film comprising a pharmaceutically active component according to claim 22 wherein said converting step comprises forming a pliable film with said active component distributed substantially homogeneously through said film.

26. A method of manufacturing a film comprising a pharmaceutically active component according to claim 22 wherein said connecting step comprises extruding said fluid mixture through an extruder.

27. A method of manufacturing a film comprising a pharmaceutically active component according to claim 26 wherein said forming step further comprises extruding said fluid mixture through an extruder comprising at least one baffle.

28. A method of manufacturing a film comprising a pharmaceutically active component according to claim 22 wherein said forming step further comprises extruding said fluid mixture through an extruder comprising at least one mixing paddle.

29. A method of manufacturing a film comprising a pharmaceutically active component according to claim 22 wherein said forming step further comprises extruding said fluid mixture through an extruder comprising at least one generally cylindrical mixing paddle.

30. A method of manufacturing a film comprising a pharmaceutically active component according to claim 22 wherein said forming step further comprises extruding said fluid mixture through an extruder comprising a plurality of mixing paddles.

31. A method of manufacturing a film comprising a pharmaceutically active component according to claim 23 wherein said forming step further comprises extruding said fluid mixture through an extruder comprising at least one vacuum for removing air from said fluid mixture.

32. Apparatus for manufacturing a film comprising at least one pharmaceutically active agent, at least one lubricant, at least one solvent for the binding agent and the lubricant, and at least one pharmaceutically active component which is not soluble in said solvent comprising:
providing at least one mixing tank for preparing a liquid suspension;
at least one chamber comprising an extruder comprising an inlet, means for mixing the liquid suspension as the liquid suspension passes through said chamber of said extruder and an exit orifice; and
a moveable belt for receiving a sheet of said suspension exiting said exit orifice.

33. An apparatus according to claim 32 wherein said extruder comprises at least two chambers comprising means for mixing said liquid suspension.

34. An apparatus according to claim 33 wherein at least one of said mixing means comprises a movable impeller.

35. An apparatus according to claim 33 wherein at least one of said mixing means comprises a movable blade.

36. An apparatus according to claim 34 wherein at least one of said mixing means comprises a plurality of baffles.

37. An apparatus according to claim 36 wherein at least one of said chambers further comprises means for removing gas from said chamber.

38. An apparatus according to claim 33 wherein at least one of said mixing means comprises a plurality of baffles.

39. An apparatus according to claim 33 wherein at least one of said chambers further comprises means for removing gas from said chamber.

40. An apparatus according to claim 32 wherein said extruder comprises at least two chambers comprising means for urging a sheet from said exit orifice toward said moveable belt.

41. Apparatus according to claim 40 wherein said urging means comprises a vacuum slot disposed below said exit orifice.

42. An apparatus according to claim 33 wherein at least one of said mixing means comprises a generally cylindrical mixing paddle.

43. A method for administering pharmaceutical actives to a patient comprising the steps of:
providing a film comprising at least one binding agent, at least one lubricant, at least one solvent for the binding agent and the lubricant, and an effective amount of at least one pharmaceutically active component which is not soluble in the solvent;
dissolving said film in at least one solvent in which the pharmaceutically active agent is soluble; and
administering the resultant solution to a patient.

44. A method according to claim 43 wherein said administering step comprises administering said solution to a patient orally.

45. A method according to claim 43 wherein said administering step comprises administering said solution to a patient as a vapor.

46. A method according to claim 43 wherein said administering step comprises administering said solution to a patient topically.

47. A method according to claim 43 wherein said administering step comprises administering said solution to a patient intravenously.

48. A method according to claim 43 wherein said administering step comprises administering said solution to a patient intramuscularly.

49. A method according to claim 43 wherein said administering step comprises administering said solution to a patient into a body cavity.

50. A method according to claim 43 wherein said step of providing a film comprises providing a film in a pouch comprising a solvent which is separated from said film by a barrier, and said dissolving step comprises breaching said barrier.
